(19) United States
(12) Patent Application Publication
(10) Pub. No.: US 2024/0293772 A1
(43) Pub. Date: Sep. 5, 2024

(54) SYSTEM AND METHOD FOR CARBON CAPTURE AND UTILIZATION

(71) Applicant: Cyprus University of Technology, Lemesos (CY)

(72) Inventor: Ioannis Vyrides, Nicosia (CY)

(73) Assignee: Cyprus University of Technology, Lemesos (CY)

(21) Appl. No.: 18/568,379

(22) PCT Filed: Jun. 9, 2021
(86) PCT No.: PCT/IB2021/055067

§ 371 Date: Dec. 8, 2023

(51) Int. Cl. B01D 53/14; C12P 5/02

(52) U.S. Cl. CPC B01D 53/1493; B01D 53/1418; B01D 53/1425; C12P 5/023; B01D 2251/304; B01D 2251/604; B01D 2257/504

(57) ABSTRACT

A system and a method for converting captured carbon dioxide (CO₂) into hydrogen (H₂) by using metallic iron or metallic magnesium in anaerobic process are described. In a first step, the CO₂ can be absorbed in an alkaline solution such as NaOH and a soluble bicarbonate is formed. In a second step, the soluble bicarbonate HCO₃— is converted into H₂ by reacting it with zero valent metal, like metallic Fe (powder) or scrap Fe or Magnesium ribbon in anaerobic ambient conditions. Metal carbonate, like siderite, is created on the outer surface of Fe(⁰) and can be separated by the alkaline solution, which is recycled in the first reaction to be used for CO₂ absorption. Exposing the separated siderite to weak acid, either citric acid or oxalic acid, zero valent metal is obtained, which is recycled in the second reaction. Alternatively, the siderite can be used as a raw material in the steel industry or cement industry or commercialized as an iron scrap. The generated H₂ can be directly used for energy purposes or can be directed to another reactor comprising also bicarbonate solution and mix hydrogenotrophic methanogens to be converted into methane (CH₄) or to a bioreactor comprising homoacetogenic bacteria to be converted into carboxylic acids, like acetic acid (CH₃COOH). Alternatively, the reaction with Fe(⁰) or Mg(⁰), bicarbonate solution, CO₂ and hydrogenotrophic methanogens for the production of CH₄ can take place in one and same bioreactor.

### FIELD OF INVENTION

The present invention relates to carbon dioxide (CO₂) capture system and method and more specifically to a system and method for the carbon dioxide (CO₂) capture and utilization (CCU), converting the captured carbon dioxide (CO₂) to chemical compounds, like hydrogen (H₂) or methane (CH₄), by using metallic iron and/or metallic magnesium in anaerobic process.

In particular, the carbon dioxide (CO₂) capture and utilization method comprises a) alkaline solution with metallic iron and/or metallic magnesium to convert carbon dioxide (CO₂) to hydrogen (H₂) or b) alkaline solution with metallic iron and/or metallic magnesium and mix hydrogenotrophic methanogens to convert carbon dioxide (CO₂) to methane (CH₄).

### BACKGROUND

Carbon dioxide (CO₂), as the most significant anthropogenic greenhouse gas (GHG), substantially contributes to global warming and climate change. By the 2016 Paris Agreement has been firmly established that CO₂ is the primary driver of global warming and, for this reason, investor pressure and voluntary responses from many multinational firms and industries have been stimulated, creating significant opportunities for technological advances. In the European Union, the industrial plants are subject to the EU Emissions Trading System and have to reduce their GHG by 43% in 2030 compared to 2005. From 2021 onwards, if EU countries do not meet their decarbonization path up to 2030, they will have to purchase emission permits to make up for the shortfall in emission reduction which will be funded by the public budget.

To mitigate this, researchers and engineers have examined processes for transforming CO₂ into commodity chemicals and fuels; the process is also known as carbon dioxide (CO₂) capture and utilization (CCU). However, this is considered a challenge, as CO₂ presents kinetic and thermodynamic stability due to its centrosymmetric structure and the use of high-energy substances or processes is required for its reduction.

The most frequent carbon dioxide capture technologies are absorption, adsorption, membrane separation, and cryogenic technology. Among them, the most mature and most widely used technology is absorption, which captures CO₂ through chemical reactions.

Monoethanolamine (MEA) process has become the most widely used process for the CO₂ capture; however, the main disadvantages of MEA in CO₂ absorption are that MEA is volatile and easily degraded, leading to a large amount of solvent loss.

Another practical method to capture CO₂ is by absorbing it into alkaline aqueous solutions, like NaOH, and producing bicarbonate. The CO₂ absorption capacity of NaOH solution is higher than that of MEA, and NaOH is even more abundant and cheaper than MEA.

For example, the US patent US 2010/0034724 A1 discloses a method of carbon dioxide capture, wherein in a step (a) anhydrous sodium carbonate is separated from a first aqueous solution formed by reacting carbon dioxide and an aqueous solution of sodium hydroxide. In step (b) the anhydrous Sodium carbonate is treated by causticization to generate carbon dioxide and sodium hydroxide. The first aqueous solution of step (a) is formed by Scrubbing a gas containing carbon dioxide with an aqueous solution of Sodium hydroxide.

For example, the European patent application EP 3,673,972 A1 is related to a method and related device for capturing carbon dioxide gas by contacting it with a liquid alkaline solution, which react with the carbon dioxide and converts it into (bi)carbonate, and including a further processing step to convert the (bi)carbonate to an economically valuable chemical compound.

For example, the international application WO 2008/099252 A1 discloses an anaerobic process for converting CO₂ into methane. In one embodiment is described a module/phase comprising the following stages:
CO₂ removal from flue gas in an extraction zone, by means of an alkaline solution of sodium minerals and CO₂ dissolution and hydration catalyzed by immobilized carbonic anhydrase;
precipitation by sodium carbonate of the species resulting from CO₂ hydration and obtainment of an oversaturated sodium bicarbonate solution [ . . . ].

The problem of capturing carbon dioxide (CO₂) by absorbing it into alkaline aqueous solutions, like NaOH, producing bicarbonate is that bicarbonate has a high solubility, is stable and is hardly decarbonized. Moreover, NaOH, can hardly be re-generate from the bicarbonate.

Several studies have demonstrated the use of bicarbonate solution as a feed for microalgae, while other research have mixed the bicarbonate solution with calcium or magnesium waste to produce CaCO₃ and MgCO₃, respectively.

See, for example, “A novel concept of bicarbonate-carbon utilization via an absorption-microalgae hybrid process assisted with nutrient recycling from soybean wastewater, Chunfeng Song, et al., Journal of Cleaner Production, Volume 237, 10 Nov. 2019.”

For example, the US patent application US 2007/0202032 A1, discloses a process for producing CaCO₃ or MgCO₃ from a feedstock comprising a Ca- or Mg-comprising mixed metal oxide, and to a process for the production of an aqueous solution of Ca(HCO₃)₂ or Mg(HCO₃)₂.

As may be understood from the cited examples, so far, a circular process that using alkaline aqueous solutions, like NaOH, can lead to the capture of CO₂, forming bicarbonate solution and producing Hydrogen, regenerating at the same time the alkaline NaOH solution, is not available from the art, and therefore is desirable.

Furthermore, large industries, like power stations, cement plants and steel plants emit vast amounts of CO₂. More specifically, iron steel industries and cement industries, apart from the huge amount of CO₂, make use of siderite as a raw material in their process. For example, magnesium carbonate is used in the cement industry.

Hence a CCU process that produces as reaction by-product metallic iron (Fe), like siderite, or magnesium carbonate, is indeed desirable because, they can be directly utilized as a raw material in steel industry.

Another potential field of application lies in the field of biogas upgrading. Biogas is the product of the degradation of organic matter in the absence of oxygen (anaerobically), and consists mainly of methane and carbon dioxide, and biogas upgrade is the process of separating methane from carbon dioxide and other gases from biogas. There are more than 17,736 biogas plants in Europe and therefore CO₂ capture and utilization from biogas increase its calorific value as well as its potential use.

There are many Carbon capture and utilization (CCU) and direct air capture (DAC) based companies that use mainly physicochemical processes. Therefore, being able to take advantage of a new low cost/efficient CCU process would be desirable for this type of business.

Finally, another potential application could be found in the generation of energy in remote places, even in spacecraft mission on Mars. For example, Mars mineral composition consists mainly of iron and magnesium minerals and the atmosphere is CO₂, and there is water-ice on its surface. Therefore, a method which exploits the abundance of carbon dioxide and of minerals on the soil of Mars could be a solution for an abundant energy generation directly on planet.

### SUMMARY

A specific object of the present invention is that of providing a system and method for capturing the carbon dioxide (CO₂) by converting it as soluble bicarbonate HCO₃-to H₂ using zero valent metal (Fe⁽⁰⁾ or Mg⁽⁰⁾) or CH₄ using hydrogenotrophic microorganism under ambient anaerobic aquatic conditions (H₂O).

In accordance with the present invention, there is thus provided a process by which carbon dioxide (CO₂) is absorbed in an alkaline solution, for example an aqueous solution comprising an alkali metal hydroxide, for example sodium hydroxide (NaOH), and soluble bicarbonate (NaHCO₃) is formed. Soluble bicarbonate (NaHCO₃) is first reacted with zero valent metal, for example commercial powder iron (Fe) or magnesium (Mg) ribbon and converted to hydrogen gas (H₂), comprising a concentration>97% H₂.

According to an aspect of the present invention, the capturing of carbon dioxide (CO₂) is used to generate hydrogen gas (H₂) for being directly used for energy purposes. According to the present invention, the reaction shows higher reaction rate for production of H₂ with higher bicarbonate concentration, for example 10-100 [gr NaHCO₃/L] compared to the same reaction with oxygen or with a lower bicarbonate concentration.

According to a first embodiment of the method, a circular process is created, and the solution after having reacted is reusable for CO₂ capture. In fact, the siderite, which is created on the outer surface of metal iron Fe⁽⁰⁾ after a first reaction, can be separated by the alkaline solution. By exposing it to citric acid (0.1M-0.5M) or other weak acids in a second reaction, the siderite in the outer surface of Fe⁽⁰⁾ is going to be removed. After this the citric acid solution is removed, and the remaining Fe⁽⁰⁾ is recyclable in the reaction for the H₂ production.

According to another embodiment, the siderite, instead of being removed from the outer surface of Fe⁽⁰⁾, it can be used as is, for example, as raw material for the steel industry or cement industry or as an iron scrap. With the same purpose, Magnesium carbonate can also be used as a raw material in the cement industry.

According to a further embodiment, the siderite can be used for other applications such as Phosphate absorption or heavy metals adsorption from wastewater.

According to another aspect of the present invention, hydrogen gas (H₂), instead of being used for energy purposes, it can be used in another reaction for the production of methane (CH₄), which has more potential final use. For example, is known the use of H₂ from electrolysis to feed the hydrogenotrophic methanogens. The hydrogenotrophic methanogens convert H₂ and soluble CO₂ to methane (CH₄). According to an embodiment of the present method, the hydrogen (H₂) generated in a first reaction is then directed in another reactor comprising bicarbonate solution and mix hydrogenotrophic methanogens. Experimental results have shown that in 4 days higher than 98% of CH₄ is obtainable by a system comprising CO₂ and Fe⁽⁰⁾ bicarbonate solution and mix hydrogenotrophic methanogens

According to still another embodiment, H₂ generated by the first reaction can be directed to a bioreactor comprising ‘homoacetogenic’ bacteria that converts H₂ and CO₂ to carboxylic acids, mainly acetic acid.

### BRIEF DESCRIPTION OF THE DRAWINGS

For a better understanding of the present invention, preferred embodiments, which are intended purely by way of example and are not to be construed as limiting, will now be described with reference to the attached drawings, where:

FIG. 1 is a diagram representing a first embodiment of the method of capturing carbon dioxide according to an aspect of the present invention.

FIG. 2 is a diagram representing a second embodiment of the method of capturing carbon dioxide according to another aspect of the present invention.

FIG. 3 is a diagram representing a third embodiment of the method of capturing carbon dioxide according to an aspect of the present invention.

FIG. 4. Production of H₂ (mmol)/Fe(kg) from Fe⁽⁰⁾ (50 gr/L) in solution (a) flushed with N₂, (b) flushed with CO₂ and (c) with 10 gr NaHCO₃/L flushed with CO₂.

FIG. 5a. Production of H₂ (mmol)/Fe(kg) from Fe⁽⁰⁾ (25 g/L) in solution (a) flushed with N₂, (b) flushed with CO₂, (c) with 25 gr NaHCO₃/L flushed with CO₂ and (d) with 50 gr NaHCO₃/L flushed with CO₂. Initial pH=7.3.

FIG. 5b. Production of H₂ (%) from Fe⁽⁰⁾ (25 g/L) in solution (a) flushed with N₂, (b) flushed with CO₂, (c) with 25 gr NaHCO₃/L flushed with CO₂ and (d) with 50 gr NaHCO₃/L flushed with CO₂. Initial pH=7.3.

FIG. 6. Production of H₂ (%) in solution with 10 gr NaHCO₃/L, flushed with CO₂ and with Fe⁽⁰⁾ concentration of 5 gr^{/}L, 10 gr/L, 25 gr/L, 50 gr/L.

FIG. 7. Production of H₂ (%) from Mg⁽⁰⁾ (2 gr/L) in solution (a) exposed to air, (b) flushed with CO₂, (c) with 10 gr NaHCO₃/L flushed with CO₂. Initial pH=6.

FIG. 8. Production of H₂ (%) over time from Mg⁽⁰⁾ (2 gr/L) in solution (a) flushed with CO₂, (b) with 10 gr NaHCO₃/L flushed with CO₂, (c) with 20 gr NaHCO₃/L flushed with CO₂ and (d) 40 gr NaHCO₃/L flushed with CO₂. Initial pH=7.3.

FIG. 9. Production of H₂ (%) over time from Fe⁽⁰⁾ (50 gr/L) in solution of NaHCO₃ previously formed from 0.35M NaOH and CO₂. Initial pH=7.3.

FIG. 10. Gibbs free energy change ΔG (KJ/mol) for metallic iron reaction with H₂O and N₂ at headspace and Gibbs free energy change ΔG (KJ/mol) for metallic iron reaction with H₂O, bicarbonate and CO₂ at headspace.

### DETAILED DESCRIPTION

The following discussion is presented to enable a person skilled in the art to make and use the invention. Various modifications to the embodiments will be readily apparent to those skilled in the art, without departing from the scope of the present invention as claimed. Thus, the present invention is not intended to be limited to the embodiments described therein, but it has to be accorded the widest scope consistent with the principles and features disclosed herein and defined in the appended claims.

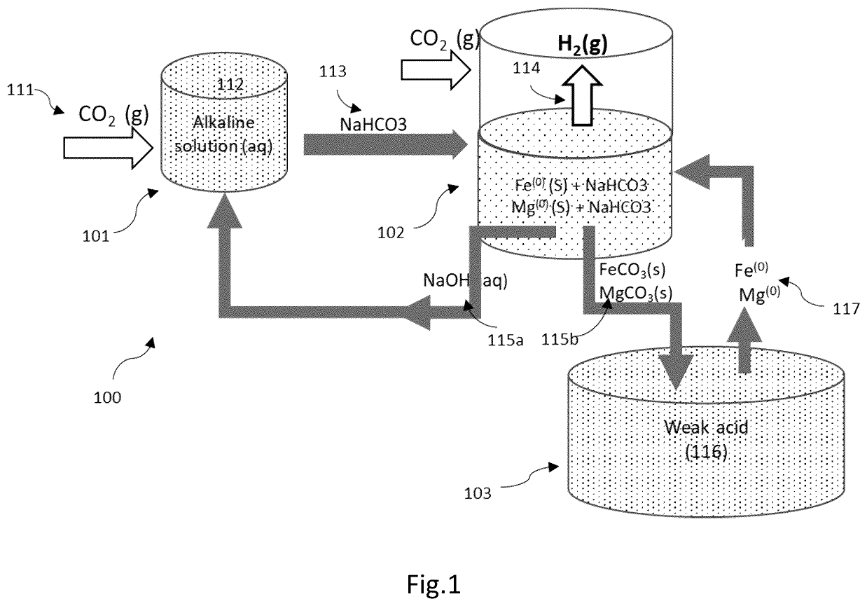

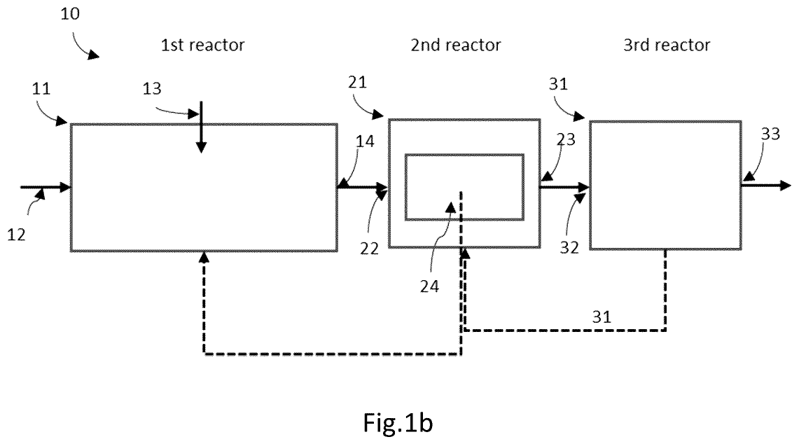

According to FIG. 1, a process (100) of capturing and converting carbon dioxide (CO₂) is described comprising a first step (101) by which carbon dioxide, CO₂ (111) is absorbed in an alkaline solution (112), typically an aqueous solution comprising, for example, sodium hydroxide (NaOH), with a concentration comprised between 0.1M to 1.3M, preferably with a concentration of 0.9M, having an initial pH>10, and as a result soluble bicarbonate, NaHCO₃ (113), having pH between 7-8, is formed, according for example to the following reaction formula (1):

NaOH (aq)+CO₂→NaHCO₃ (aq) 1)

Bicarbonate (NaHCO₃), as reaction product, is stable and is hardly decarbonized. To overcome this problem, a second step (102) is used, wherein bicarbonate (113) is reacted with zero valent metal, for example commercial powder Fe, to produce hydrogen gas, H₂ (114), according for example to the reaction formula (2):

Fe⁽⁰⁾ (s)+HCO₃— (aq)+H₂O→FeCO₃ (s)+H₂ (g)+OH (aq). 2)

As shown in FIG. 1, in step (102) the bicarbonate solution reacts with metallic Fe⁽⁰⁾ and H₂ (114) is generated in the headspace. Also, the initial CO₂ in the headspace is moved to the liquid solution and reacts with metallic Fe⁽⁰⁾ therefore the headspace contains final >98% H₂. As shown by the reaction formula (2), the second reaction step (102) comprises the production of solid metal carbonate, for example FeCO₃, as by-product increasing alkalinity (OH⁻).

Instead of commercial powder Fe, a scrap-Fe can be used, which will significantly reduce the process cost; however, the reaction rate is 4-5 times slower but the final H₂ concentration is still more than 98% Furthermore, Magnesium (Mg⁽⁰⁾) ribbon can be used instead of Fe⁽⁰⁾ at a lower concentration than Fe, according to the reaction formula (3):

Mg⁽⁰⁾ (s)+HCO₃— (aq)+H₂O→MgCO₃ (s)+H₂ (g)+OH— (aq). 3)

Both the reactions (2) and (3) occur in anaerobic carbonate ambient conditions using metallic Fe (powder) or scrap Fe or Magnesium (Mg) ribbon.

According to an aspect of the present invention, the reaction become faster (higher reaction rate) in the production of H₂ in the presence of high concentration of bicarbonate, for example (10-100 gr NaHCO₃/L). Final gas composition comprises a Hydrogen (H₂) concentration>98% after a reaction time ranging from 1 to 96 hours.

The rate and the yield of reaction depend on several conditions, comprising:
Fe or Mg concentration,
surface area of Fe or Mg,
bicarbonate concentration,
temperature,
initial pH,
initial headspace gas,
agitation,
initial pressure,
headspace volume.

According to an embodiment of the present method, the reaction is more thermodynamically favorable (compared to the same conditions with oxygen or with low bicarbonate) and increases the reaction rate. Specifically using Fe⁽⁰⁾ powder, the reaction rate (H₂ mmol/Fe(kg)·(h)) of H₂ is 1.5-2.5 higher at higher bicarbonate concentration compared to the condition without bicarbonate or to the use of only CO₂. On the other hand, the reaction rate (H₂ mmol/Fe(kg)·(h)) of high bicarbonate is 250-350 higher compared with the Fe⁽⁰⁾ powder exposed to water and nitrogen (N₂). For example, the reaction (2) is very slow when N₂ is used instead of CO₂. However, bicarbonate solution and CO₂ in the gas phase (at initial conditions) result in a significantly higher H₂ production rate than using water and CO₂ in the gas phase. In this process according to reaction (2), no external energy is needed for the reaction to occur.

At the end of the reactions (2) or (3), pH is alkaline (pH 8.5-9.5), therefore once the alkaline solution is separated from Fe or Mg, it can be recycled to be used again for more CO₂ absorption as in reaction (1) and then the final pH will be drop to 7.0-8.

According to a first aspect of the present invention, the hydrogen gas, H₂ (114) generated according to the reaction formula (2) can be directly utilized for energy purpose.

According to an embodiment of the present method the metal carbonate (115b) obtained in the reaction step (102) is separated obtaining an aqueous alkaline solution, comprising for example NaOH (115a), wherein said aqueous alkaline solution is recycled in the reaction step (101). According to an embodiment of the present method, a solid metal carbonate (115b), for example the siderite (FeCO₃), is created in the outer surface of Fe⁰.

The solid metal carbonate (115b) can be removed, by a further a step (103), by which weak acid (116), comprising citric acid or oxalic acid, is used with concentration in the range 0.1M-0.5M, so that the remaining zero valent metal (117) can be recycled in the reaction step (102) and the reaction according to the formula (2) can be initiated again. The generation of carbonate (115) on the surface of Fe or Mg is indicated by the reduction of the concentration of H₂ from the gas phase to lower than 95% (after several batch cycles). According to a preferred embodiment, after removal of carbonate the remaining solid (FeCO₃ or MgCO₃) is exposed to citric acid or weak acid for several hours so the external FeCO₃ is removed and the inner Fe can react again in step (102).

According to another embodiment of the present method, the solid FeCO₃ (s) or MgCO₃ (s) instead of being directed to step (103) to be removed from the outer surface, it is used as a raw material in steel industry or cement industry or they are commercialized as iron scrap.

According to a further embodiment of the present method, the siderite is used in other applications, such as Phosphate absorption or heavy metals adsorption from wastewater.

According to a second aspect of the present invention, the hydrogen gas (H₂) generated according to the reaction formula (2) or (3), instead to be used for energy purposes, is directed to a bioreactor comprising Hydrogenotrophic methanogens for methane (CH₄) production or Homoacetogenic bacteria for acetic acid (CH₃COOH) production. The bioreactor should also contain sodium bicarbonate (NaHCO₃) solution from reaction (1).

The microbial inoculum can be a mixed culture such as anaerobic granular sludge or anaerobic sludge from the digester. The inoculum can be pre-exposed to favorable conditions to enrich the hydrogenotrophic methanogens or homoacetogens, depending on the final product.

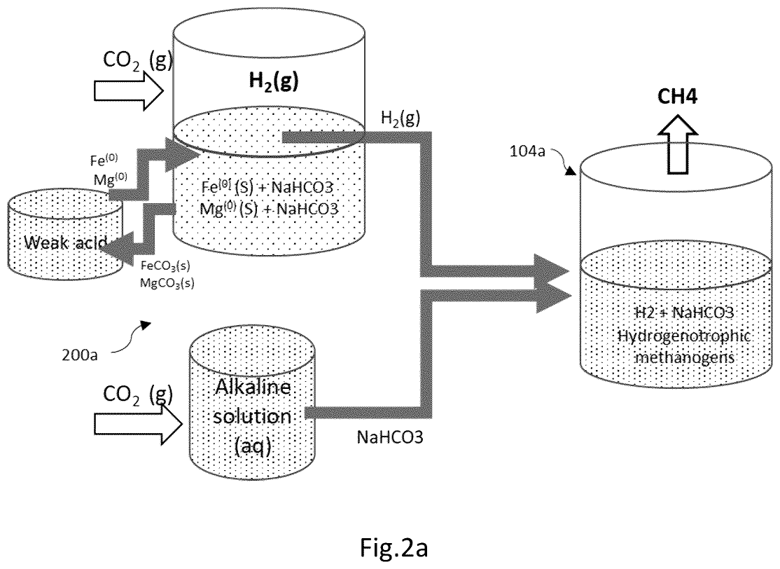

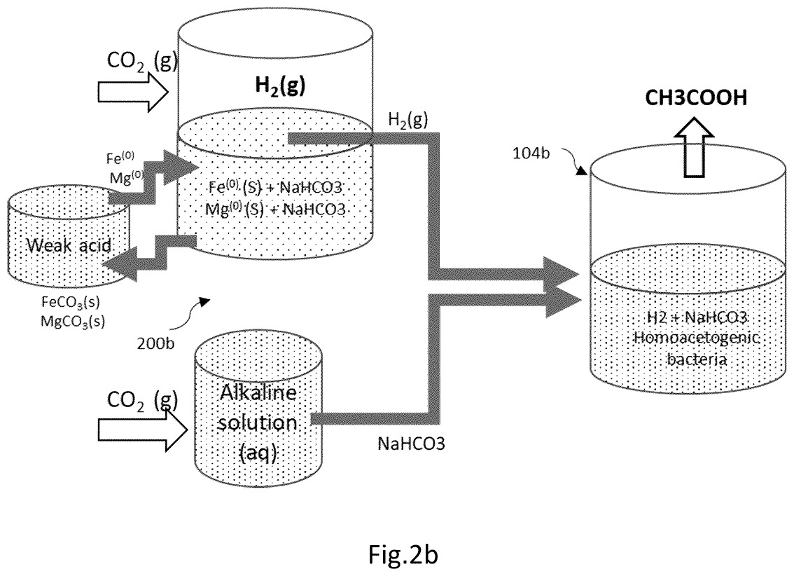

According to an embodiment of the present method (200a), the hydrogen gas (H₂) generated according to the reaction formula (2) or (3), is directed to a bioreactor (104a) comprising hydrogenotrophic methanogens, as shown in FIG. 2a, wherein the reagents H₂ and soluble CO₂ are converted to CH₄, according to a reaction formula (4):

3H₂+HCO₃→CH₄+3H₂O 4)

Methane (CH₄) has more potential final uses than H₂, said uses comprising energy, fuel vehicle, storage in natural gas grid.

Conventionally, in the process of using hydrogenotrophic methanogens to convert CO₂ to CH₄, the external H₂ gas is added from a separate electrochemical process unit (electrolysis), which splits water into H₂ gas and O₂ by utilizing surplus renewable electric power. One of the current drawbacks to use this process is the relatively high cost of electrolysis and the integration of various systems such as electrolysis process and the H₂ addition to anaerobic digester/bioreactor. In other studies, microbial electrosynthesis has been used for biogas upgrading; however, the main challenges in this case are the cost reduction and the absence of pilot-scale and full-scale demonstration for the process.

According to the present invention, in the process of using hydrogenotrophic methanogens to convert CO₂ to CH₄, the use of H₂ as generated from the reaction (2) and/or (3) is proposed. By experimental results, it has been shown that in 4 days higher than 98% of CH₄ can be obtained starting by a system comprising CO₂ and Fe^{(o)}.

Alternatively, according to another embodiment of the present method (200b) the gaseous hydrogen (H₂) can be directed to a bioreactor (104b, FIG. 2b) comprising homoacetogenic bacteria to convert H₂ and CO₂ to carboxylic acids, for example acetic acid (CH₃COOH) according to a reaction formula (5):

2HCO₃—+4.5H₂→CH₃COO—+4H₂O 5)

At the end of reaction (4) or reaction (5) pH is alkaline and the solution can be recycled for CO₂ absorption (as previously descripted).

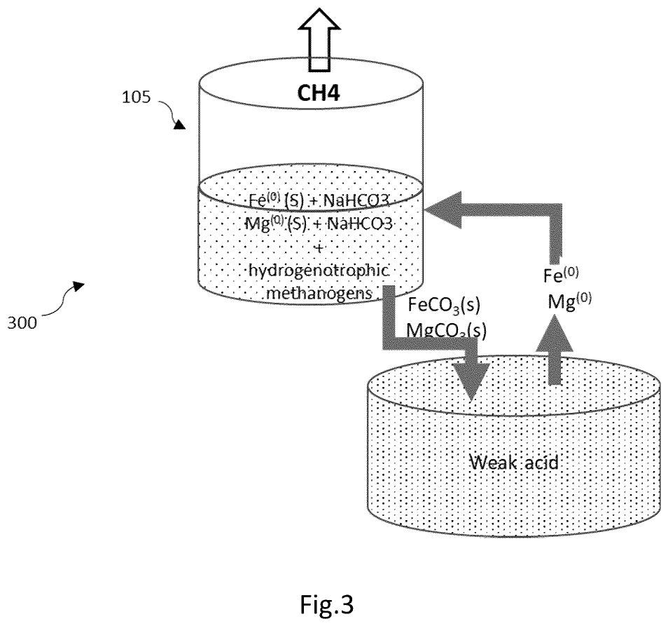

According to another embodiment (300) of the present method, the reactions (2) or (3) can take place “in-situ” with the reaction (4) in one and the same reactor (105, FIG. 3), reacting them with hydrogenotrophic methanogens in the same reactor, in such a way to produce a final gas with CH₄>97%, according to the reaction formula (6) or (7):

4Fe⁽⁰⁾+4HCO₃+4H⁺+CO₂→4FeCO₃+CH₄+2H₂O; 6)

Mg⁽⁰⁾+4HCO₃+4H⁺+CO₂→4MgCO₃+CH₄+2H₂O. 7)

The limitation of using this “in-situ” process is the inhibition of sodium to the microorganisms; therefore, the sodium bicarbonate should be less than 30 gr NaHCO₃/L. For higher concentrations of sodium bicarbonate, halophilic microorganisms has to be used.

The FeCO₃ formation can be removed with citric acid as already shown in the foregoing. The reaction (6) or (7) will require about 3-6 days to be completed (CH4>97%).

Referring to FIG. 1b, an apparatus (10) for carrying out the method of capturing and converting carbon dioxide (CO₂) as described in the foregoing comprises at least a first reactor (11) and a second reactor (21). The first reactor (11) comprises a first inlet module (12) for the gaseous carbon dioxide (CO₂), a second inlet module (13) for an aqueous alkaline solution comprising an alkali metal hydroxide, at least one outlet module (14), and is configured to absorb the gaseous carbon dioxide in the aqueous alkaline solution comprising an alkali metal hydroxide such that the carbon dioxide reacts with the alkali-metal hydroxide to form a bicarbonate. The second reactor (21) comprises an inlet module (22) for the bicarbonate solution, at least one outlet module (23), and is operatively connected to said first reactor (11) and is configured to bring in contact the bicarbonate solution with a zero valent metal species to form a reaction mixture of H₂, solid metal carbonate and aqueous alkaline solution.

According to a preferred embodiment of said apparatus (10), said second reactor (21) comprises a liquid/solid separation module (24) configured to separate the reaction mixture into a regenerated aqueous alkaline solution and a solid metal carbonate, wherein said regenerated aqueous alkaline solution is recycled in the first reactor (11).

According to an embodiment, said apparatus comprises a third reactor (31), having an inlet module (32) and at least one outlet module (33), and is configured to receive Fe carbonate from the second reactor (21), bring in contact said Fe carbonate with either citric acid or oxalic acid to form zero valent Fe, wherein said zero valent Fe is recycled in the second reactor (21).

### Experimental Results

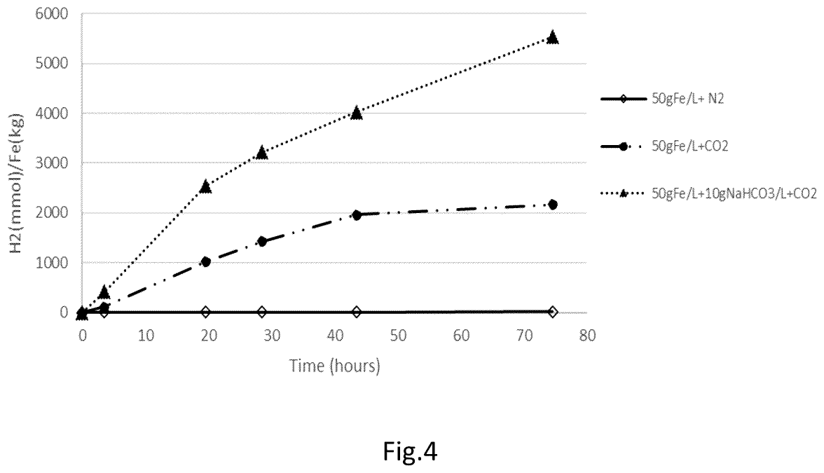

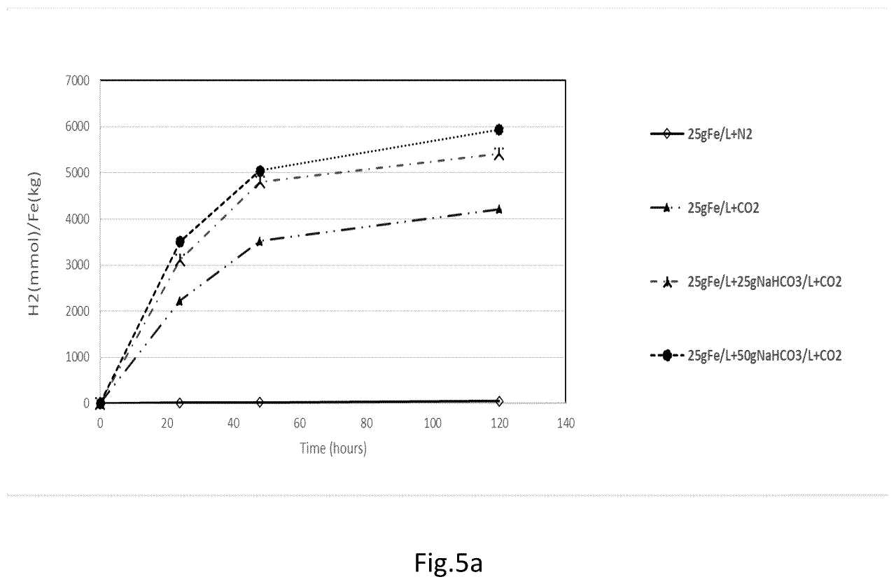

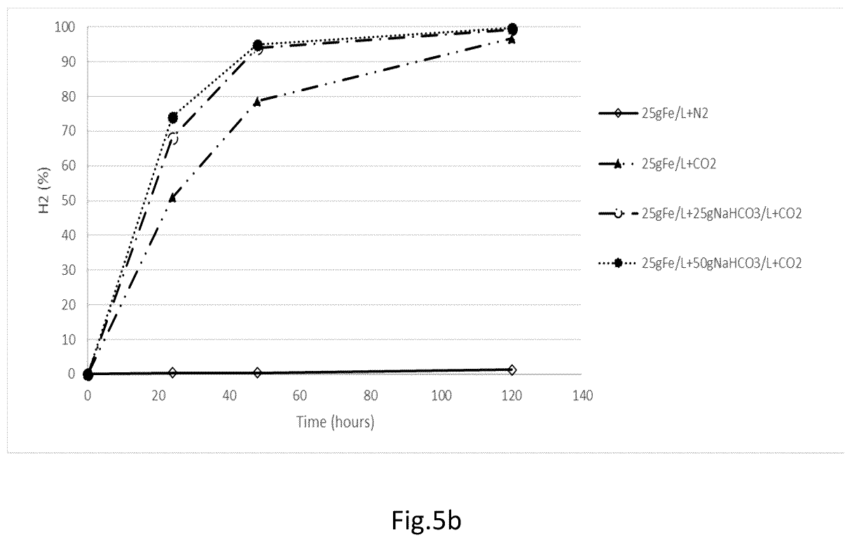

1) Examined parameters: N₂, CO₂ and 10 gr NaHCO₃/L. with CO₂ at 50 gr Fe⁽⁰⁾/L.
   Experimental condition: Fe⁽⁰⁾ (50 g/L), serum bottles (165 ml), working volume 65 ml of water, headspace 100 ml, temperature 33° C., initial pH 6, agitation 100 rpm.
      As can be seen from the chart in FIG. 4, representing the H₂ mol generation Vs time, when the system was flushed with N₂ (for 2 min), it resulted in negligible H₂ production after 75 hours. On the contrary, when the system was a) flushed with CO₂ or b) with CO₂ plus 10 gr NaHCO₃/L in the solution, it resulted in a dramatically higher H₂ (mmol) generated over time. The presence of 10 gr NaHCO₃/L in addition to being flushed with CO₂ resulted in double H₂ mmol/Fe(kg) than in the samples flushed with CO₂ without extra 10 gr NaHCO₃/L.
2) Examined parameters: N₂, CO₂, 25 gr NaHCO₃/L with CO₂ and 50 gr NaHCO₃/L with CO2 at 25 gr Fe⁽⁰⁾/L.
   Experimental condition: Fe⁽⁰⁾ (25 gr/L), serum bottles (165 ml), working volume 50 ml of water, headspace 115 ml, temperature 33° C., initial pH 7.3, agitation 100 rpm.
3) The chart in FIG. 5a shows that the higher the bicarbonate concentration, the higher the H₂(mmol)/Fe(kg) generated over time. Again, the use of N₂ instead of CO₂ results in negligible H₂ production FIG. 5a and FIG. 5b. The samples with bicarbonate solution (25 and 50 gr NaHCO₃/L) produced higher than 94% H₂ in 48 hours, whereas the samples flushed with CO₂ without the addition of NaHCO₃ generated 78% H₂.
   Examined parameters: N₂, CO₂, 25 gr NaHCO₃/L with CO₂ and 50 gr NaHCO₃/L with CO₂, 75 gr NaHCO₃/L with CO₂, 100 gr NaHCO₃/L with CO₂ at 25 gFe⁽⁰⁾/L.
   Experimental conditions: Fe⁽⁰⁾ (25 g/L), serum bottles (165 ml), working volume 50 ml of water, headspace 115 ml, temperature 33° C., initial pH 7.5-7.7, agitation 100 rpm.
   Table 1 shows the production of H₂ (mmol)/Fe(kg)·h from Fe⁽⁰⁾ (25 gr/L) under various NaHCO₃ concentrations with a) flushed with N₂ without NaHCO₃ b) flushed with CO₂ without NaHCO₃ c) 25 gr NaHCO₃/L and flushed with CO₂ d) 50 gr NaHCO₃/L and flushed with CO₂.

| | Rate of the reaction after 24 h |
|---|---|
| | H2 (mmol)/Fe(kg) · h |
| Fe(0) exposed to N₂ | 0.22 |
| Fe(0) exposed to CO₂ | 101.7 |
| Fe(0) exposed to 25 g NaHCO₃/L + CO₂ | 151.5 |
| Fe(0) exposed to 50 g NaHCO₃/L + CO₂ | 160.8 |
| Fe(0) exposed to 75 g NaHCO₃/L + CO₂ | 173.1 |
| Fe(0) exposed to 100 g NaHCO₃/L + CO₂ | 198.8 |

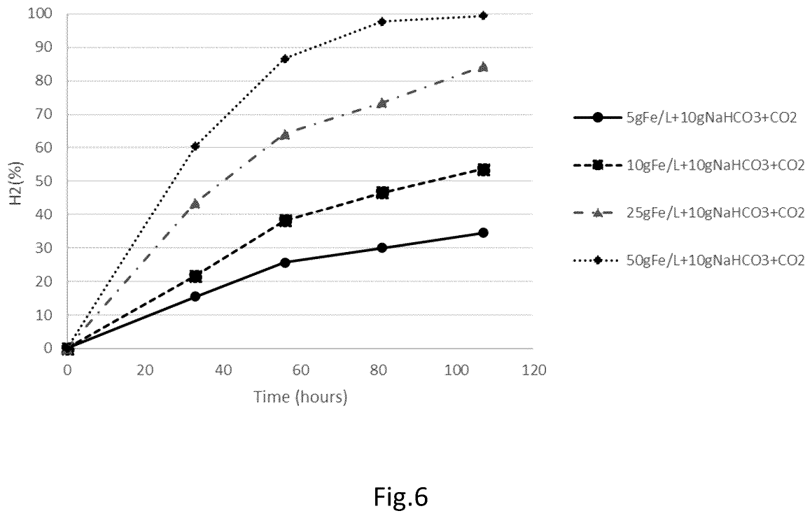

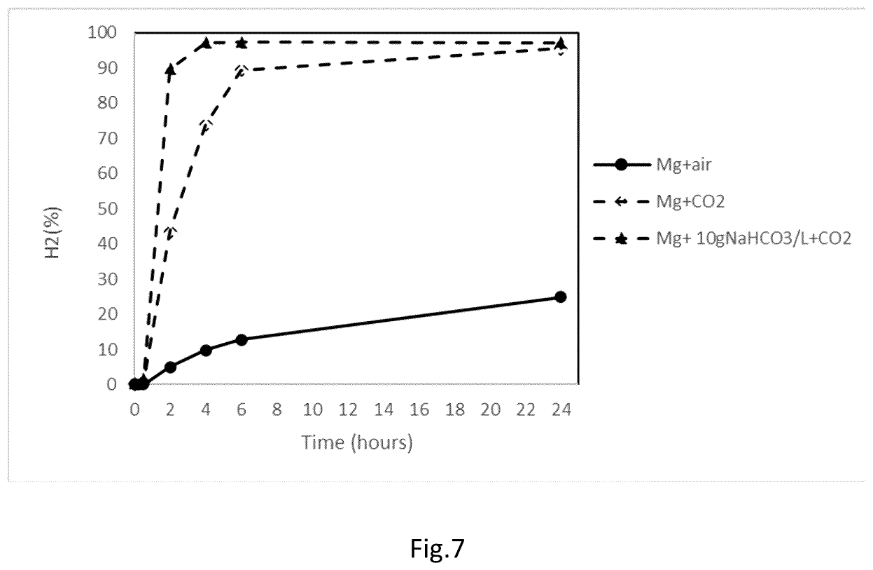

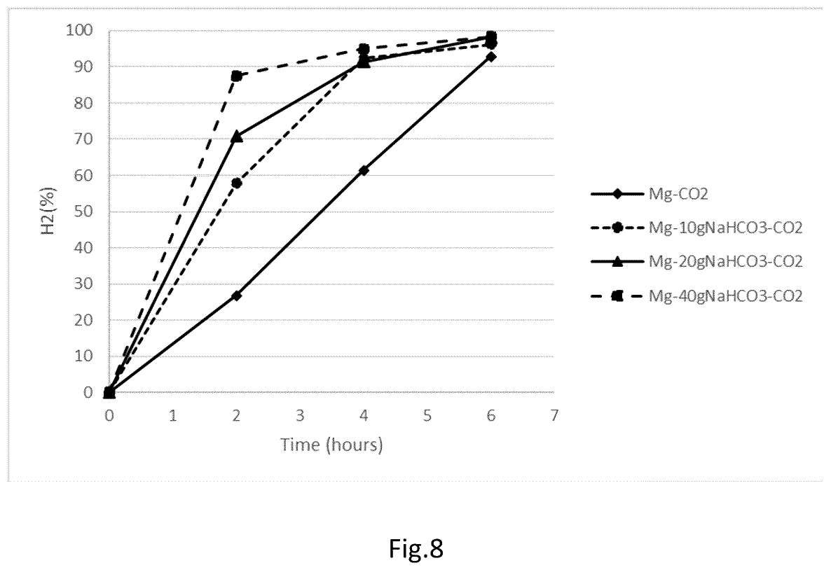

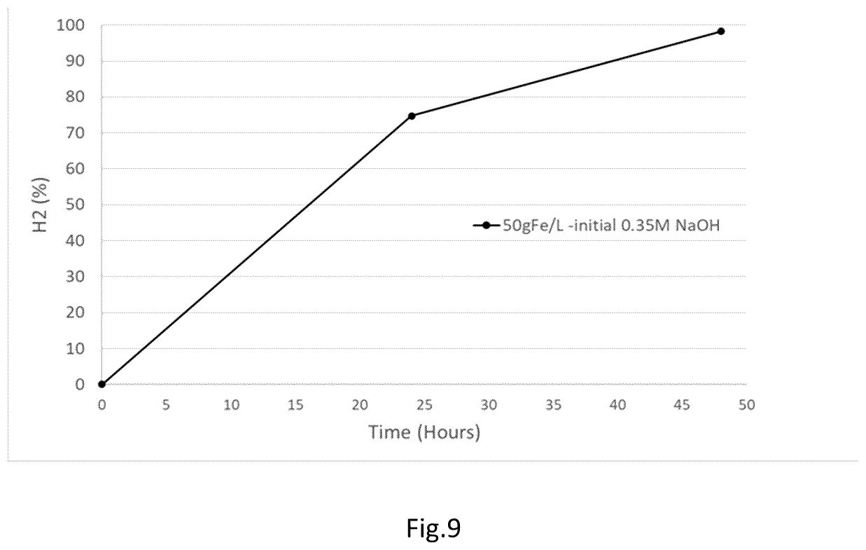

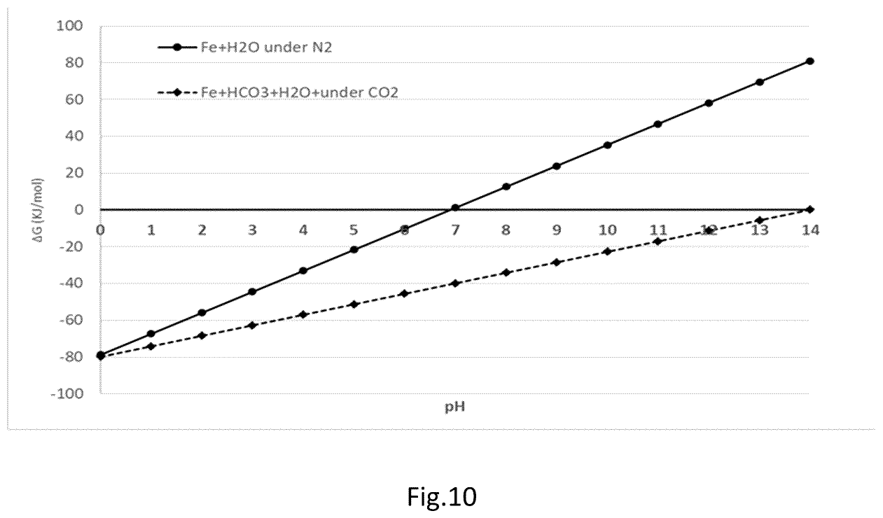

The rate H₂ (mmol)/Fe(kg)·h of the reaction for Eq 2a for Fe⁽⁰⁾ was measured after 24 h. The higher the bicarbonate concentration, the higher the rate for higher production, as shown in Table 1. In the samples flushed with CO₂, the presence of 100 gr NaHCO₃/L almost double the reaction rate compared with no extra NaHCO₃ added in the solution.
4) Examined parameters: Concentrations of Fe⁽⁰⁾ (5, 10, 25, 50 gr/L) under 10 gr NaHCO₃ and CO₂.
   Experimental conditions: Fe⁽⁰⁾ (50 g/L), serum bottles (165 ml), working volume 65 ml of water, headspace 100 ml, temperature 33° C., initial pH 6, agitation 100 rpm.
   Results from section FIG. 6 show that the higher concentrations of powder Fe resulted in higher production of H₂. At 50 gr Fe/L with 10 gr NaHCO₃/L, the H₂ is higher than 97% after 81 hours, whereas at this time point for 25 gr Fe/L, the H₂ concentration was 84%. However, as shown in FIG. 5b, the samples with 25 gr Fe/L and 25 gr NaHCO₃/L generated higher than 94% H₂ in 48 h. With 50 g/L Fe⁽⁰⁾ and 75 gr/L NaHCO₃ higher than 92% and 99% H₂ in 24 and 48 hours, are respectively produced. Therefore, both the Fe⁽⁰⁾ and NaHCO₃ positively contribute to H₂ production in the reaction, although NaHCO₃ concentrations had a more profound positive effect than Fe.
5) Examined parameters: exposure to Magnesium ribbon in air, in CO₂ ambient and CO₂+NaHCO₃ ambient.
   Experimental conditions: Mg⁽⁰⁾ ribbon (2 gr/L), serum bottles (125 ml), working volume 70 ml of water, headspace 55 ml, temperature 33° C., initial pH 6, agitation 100 rpm.
   As shown in FIG. 7, after 2 hours, the presence of bicarbonate and CO₂ resulted in a higher rate of H₂ production (around 90% H₂), whereas the exposure of the samples to CO₂ resulted in 43% H₂ in 24 hours.
6) Examined parameters: exposure to Magnesium ribbon to CO₂, CO₂+10 g NaHCO₃/L, CO₂+20 g NaHCO₃/L and CO₂+40 gNaHCO₃/L.
   Experimental conditions: Mg⁽⁰⁾ ribbon (2 gr/L), serum bottles (125 ml), working volume 70 ml of water, headspace 55 ml, temperature 33° C., initial pH 7.2, agitation 100 rpm.
      As shown in FIG. 8, the higher the amount of bicarbonate, the higher the H₂ production over time. After 24 hours a crystal was formed on the metal surface. The crystal was identified to be a carbonate called Nesquehonite (MgCO₃·3H₂O).
7) Examined parameters: temperature (T)=4° C., 20° C., 33° C.
   Experimental conditions: same as experiment n.6 with 10 gr NaHCO₃/L.
   The reaction rate was:
      2856.4 H₂ mmol/Fe(Kg)·hours at T=4° C.,
      4314.4 H₂ mmol/Fe(Kg)·hours at T=20° C.,
      4746.9 H₂ mmol/Fe(Kg)·hours at T=33° C.
   At 4° C. the reaction rate is decreased however a substantial amount of H₂ can be generated.
8) Examined parameters: Concentrations of Fe⁽⁰⁾ (50 gr/L), NaHCO₃ formed from initial 0.35 M NaOH continuously flushed with CO₂.
Experimental conditions: Fe⁽⁰⁾ (50 g/L), serum bottles (125 ml), working volume 80 ml of water, headspace 45 ml, temperature 33° C., initial pH 7.3, agitation 100 rpm.
Results from section FIG. 9 show that the H₂ is higher than 98% in 48 hours. At higher initial NaOH and higher initial Fe⁽⁰⁾ concentration the reaction occurs at a higher rate.
   Examined parameters: metallic iron reaction with H₂O and N₂ at headspace by varied pH 0-14 conditions (temperature 25° C., all concentrations 1 M and all partial pressures 1 atm). Gibbs free energy change, ·G (KJ/mol) is shown in FIG. 10. The reaction (formula 8)

Fe⁽⁰⁾+2H₂O→Fe²⁺+H₂+2OH⁻ 8)
becomes unfavorable at pH higher than 7.
In FIG. 10, the Gibbs free energy change ΔG (KJ/mol) for metallic iron reaction (formula 2) with H₂O, bicarbonate and CO₂ at varied pH 0-14 (temperature 25° C., all concentrations 1 M and all partial pressures 1 atm) is also shown. In the presence of bicarbonate the reaction (formula 2) takes place even at alkaline pH.

Finally, it is clear that numerous modifications and variants can be made to the present invention, all falling within the scope of the invention, as defined in the appended claims.

1. A process for capturing and converting carbon dioxide (CO₂), the process comprising:
absorbing a flux of gaseous carbon dioxide (CO₂) in an aqueous alkaline solution comprising an alkali metal hydroxide, wherein the carbon dioxide (CO₂) reacts with the alkali metal hydroxide to form a bicarbonate, obtaining a bicarbonate solution,
converting the bicarbonate solution into hydrogen (H₂), wherein said bicarbonate in the bicarbonate solution reacts with a zero valent metal species under anaerobic conditions to produce said hydrogen gas (H₂) and an exhaust alkaline solution, wherein a solid metal carbonate is produced on the surface of the zero valent metal species as by-product, and
separating said metal carbonate and obtaining an aqueous alkaline solution, said aqueous alkaline solution being recycled in the absorbing of the flux of gaseous carbon dioxide in the aqueous alkaline solution.

2. (canceled)

3. The process of claim 1, wherein said aqueous alkaline solution is an alkaline solution containing NaOH with a concentration in a range between 0.1M to 1.3M, preferably with a concentration of 0.9M.

4. The process of claim 1, wherein said bicarbonate solution has a pH in a range between 7 and 8.

5. The process of claim 1, wherein said zero valent metal species is selected from a group comprising metallic Fe, scrap Fe, metallic Mg, Mg ribbon.

6. The process of claim 1, wherein said solid metal carbonate is Fe carbonate or Mg carbonate.

7. (canceled)

8. The process of claim 1, wherein converting the bicarbonate solution into hydrogen (H₂) further comprises treating said separated metal carbonate with either citric acid or oxalic acid to obtain zero valent metal, wherein said zero valent metal is recycled.

9. The process of claim 1, further comprising directing said hydrogen gas H₂ to a second reactor comprising a bicarbonate solution with Hydrogenotrophic methanogens to produce methane (CH₄).

10. The process of claim 1, further comprising directing said hydrogen gas H₂ to a second reactor comprising a bicarbonate solution with Homoacetogenic bacteria to produce acetic acid (CH₃COOH).

11. The process of claim 1, wherein said converting the bicarbonate solution into hydrogen (H₂) occurs in one and same reactor comprising a bicarbonate solution including zero valent metal species and Hydrogenotrophic methanogens to produce methane (CH₄).

12. An apparatus for capturing and converting carbon dioxide (CO₂), the apparatus comprising:
a first reactor comprising a first inlet module for gaseous carbon dioxide, a second inlet module for an aqueous alkaline solution comprising an alkali metal hydroxide, at least one outlet module configured to dissolve the gaseous carbon dioxide in the aqueous alkaline solution comprising an alkali metal hydroxide such that the carbon dioxide reacts with the alkali-metal hydroxide to form a bicarbonate,
a second reactor comprising an inlet module for the bicarbonate solution and at least one outlet module, wherein said second reactor is operatively connected to said first reactor and is configured to bring in contact the bicarbonate solution with a zero valent metal species under anaerobic condition to form a reaction mixture of H₂, solid metal carbonate and aqueous alkaline solution,
wherein said second reactor comprises a liquid/solid separation module configured to separate the reaction mixture into a regenerated aqueous alkaline solution and a solid metal carbonate, wherein said regenerated aqueous alkaline solution is recycled in the first reactor.

13. (canceled)

14. The apparatus of claim 12, further comprising a third reactor having an inlet module and at least one outlet module, said third reactor being configured to receive Fe carbonate from the second reactor, and bring in contact said Fe carbonate with either citric acid or oxalic acid to form zero valent Fe, wherein said zero valent Fe is recycled in the second reactor.

15. (canceled)

16. (canceled)

17. (canceled)
